(19) United States
(12) Patent Application Publication
(10) Pub. No.: US 2024/0252088 A1
(43) Pub. Date: Aug. 1, 2024

(54) HANDHELD ELECTROENCEPHALOGRAPHY DEVICE

(71) Applicant: Wave Neuroscience, Inc., Newport Beach, CA (US)

(72) Inventor: James William Phillips, Fountain Valley, CA (US)

(21) Appl. No.: 18/418,108

(22) Filed: Jan. 19, 2024

Related U.S. Application Data
(60) Provisional application No. 63/480,915, filed on Jan. 20, 2023.

(51) Int. Cl. A61B 5/291; A61B 5/00; A61B 5/15; A61B 5/256; A61B 5/273

(52) U.S. Cl. CPC A61B 5/291; A61B 5/0006; A61B 5/150809; A61B 5/256; A61B 5/273; A61B 2560/0214

(57) ABSTRACT

A device for measuring electroencephalography (EEG) recordings of a subject, the portable device including a casing, at least two first electrodes extending from a first surface of the casing, the at least two first electrodes being configured to contact a scalp of the subject during an EEG recording, and at least one second electrode disposed on a second surface of the casing and configured to provide a ground connection during the EEG recording.

### CROSS-REFERENCE TO RELATED APPLICATIONS

This application claims priority to and the benefit of U.S. Provisional Patent Application No. 63/480,915, titled “HANDHELD ELECTROENCEPHALOGRAPHY DEVICE” and filed on Jan. 20, 2023, the entire contents of which is hereby incorporated by reference herein.

### FIELD

The disclosure relates to systems and methods for measuring, collecting, and recording electroencephalography (EEG) data using a handheld and/or portable device.

### BACKGROUND

Electroencephalography (EEG) recording is often used to diagnose and monitor a number of conditions affecting the brain. EEG data is used to detect seizures, and to identify issues with memory or comprehension. EEG data is used to analyze, evaluate, and/or diagnose dementia, head injury, concussion, brain tumors, encephalitis, sleep disorders, and epilepsy.

EEGs are recorded using electrodes placed on or near the scalp of a person, generally in a configuration that covers most of the cortical surface. A typical configuration is the 10-20 system, which describes the location of scalp electrodes in the context of an EEG examination. The configuration of the 10-20 system allows for standardized testing methods to used.

### SUMMARY

The disclosure relates generally to systems and methods for measuring, collecting, and recording EEG data using a handheld and/or portable device.

It is to be understood that any combination of features from the methods disclosed herein and/or from the systems and/or devices disclosed herein may be used together, and/or that any features from any or all of these aspects may be combined with any of the features of the embodiments and/or examples disclosed herein to achieve the benefits as described in this disclosure.

At least one aspect of the present disclosure is directed to a device for measuring electroencephalography (EEG) recordings of a subject. The device includes a casing, at least two first electrodes extending from a first surface of the casing, the at least two first electrodes being configured to contact a scalp of the subject during an EEG recording, and at least one second electrode disposed on a second surface of the casing and configured to provide a ground connection during the EEG recording.

In some embodiments, the at least two first electrodes includes at least one sensing electrode and at least one reference electrode. In some embodiments, the at least one sensing electrode is configured to contact the scalp at a desired location for the EEG recording. In some embodiments, the at least one reference electrode is configured to contact the scalp at a location of low electrical activity. In some embodiments, the second surface of the casing is opposite the first surface of the casing. In some embodiments, the at least one second electrode is configured to contact a hand of the subject while the subject is holding the device. In some embodiments, the device includes a strap connected across the second surface of the casing, wherein the strap allows the hand of the subject to hold the device while contacting the at least one second electrode.

In some embodiments, the device includes a strap including a third electrode and at least one wire electrically connected to the third electrode. In some embodiments, the at least one wire is electrically connected to the at least one second electrode forming an electrical connection between the at least one second electrode and the third electrode. In some embodiments, the strap is configured to be worn such that the third electrode contacts the subject's forehead. In some embodiments, the strap is configured to be worn such that the third electrode contacts the subject's chin.

In some embodiments, the device includes a first strap connected to the second surface of the casing and a second strap connected to the second surface of the casing, wherein the first and second straps are configured to be pulled on by the subject to hold the device against the scalp of the subject. In some embodiments, one of the first strap and the second strap includes a third electrode and at least one wire electrically connected to the third electrode, wherein the at least one wire is electrically connected to the at least one second electrode forming an electrical connection between the at least one second electrode and the third electrode. In some embodiments, a hand of the user contacts the third electrode while pulling on the first strap or the second strap.

In some embodiments, the device includes a first strap including a third electrode and at least one first wire electrically connected to the third electrode and a second strap including a fourth electrode and at least one second wire electrically connected to the fourth electrode. In some embodiments, the at least one first wire is electrically connected to the at least one second electrode forming an electrical connection between the at least one second electrode and the third electrode, and the at least one second wire is electrically connected to the at least one second electrode forming an electrical connection between the at least one second electrode and the fourth electrode. In some embodiments, the first and second straps are configured to be pulled on by the subject to hold the device against the scalp of the subject. In some embodiments, a first hand of the user contacts the third electrode while pulling on the first strap and a second hand of the user contacts the fourth electrode while pulling on the second strap.

In some embodiments, the device includes at least one audio speaker configured to provide audio feedback to the subject. In some embodiments, the audio feedback provides an indication that the EEG recording has started and/or an indication that the EEG recording has ended. In some embodiments, the audio feedback provides an indication of the device's location relative to a desired location on the scalp of the subject. In some embodiments, the device includes at least one battery. In some embodiments, the at least one battery is rechargeable.

In some embodiments, the device includes at least one processor. In some embodiments, the at least one processor is configured to send data associated with the EEG recording to at least one external device. In some embodiments, the at least one processor is configured to determine at least one EEG characteristic of the subject based on the EEG recording.

### BRIEF DESCRIPTION OF THE DRAWINGS

The accompanying drawings, which are included to provide a better understanding of the disclosure, illustrate embodiments of the disclosure and together with the description serve to explain the principle of the disclosure. In the drawings:

FIG. 1 is a diagram of an EEG system in accordance with aspects described herein;

FIG. 2A-2C are diagrams of EEG systems in accordance with aspects described herein;

FIG. 3 is a diagram of a subject using an EEG system in accordance with aspects described herein;

FIG. 4 is diagram of an EEG system in accordance with aspects described herein;

FIG. 5 is a diagram of a user using an EEG system in accordance with aspects described herein;

FIG. 6 is a diagram of a user using an EEG system in accordance with aspects described herein;

FIG. 7 is diagram of an EEG system in accordance with aspects described herein;

FIG. 8 is a diagram of a user using an EEG system in accordance with aspects described herein;

FIG. 9 is diagram of an EEG system in accordance with aspects described herein; and

FIG. 10 is diagram of an amplifier system in accordance with aspects described herein.

### DETAILED DESCRIPTION

The disclosure relates to measuring, collecting, and recording electroencephalography (EEG) data using a handheld and/or portable device.

As discussed above, EEG recordings are often used to diagnose and monitor a number of conditions affecting the brain. EEG data is used to detect seizures, and to identify issues with memory or comprehension. EEG data is used to analyze, evaluate, and/or diagnose dementia, head injury, concussion, brain tumors, encephalitis, sleep disorders, and epilepsy.

EEGs are recorded using electrodes placed on or near the scalp of a person, generally in a configuration that covers most of the cortical surface. A typical configuration is the 10-20 system, which describes the location of scalp electrodes in the context of an EEG examination. The configuration of the 10-20 system allows for standardized testing methods to used.

EEGs are recorded using one or more sensing electrodes placed near particular regions of the brain. Such regions of the brain are labeled pre-frontal, frontal, temporal, parietal, occipital, and central. Each sensing electrode corresponds to a different EEG channel. A reference electrode is generally placed near brain regions of lower electrical activity, such as, for example, the midline sagittal plane of the skull. The EEG signal from a specific channel is generally the electrical potential difference between an active electrode and the reference electrode. In one example, the reference electrode is placed along the midline of the brain. In another example, the reference electrode is placed in an area near an ear of the subject, but still close to the active electrode(s).

Ground electrodes are used in areas separate from the brain such as, for example, an earlobe of the subject. In such cases, an ear clip electrode is used. The ground electrode is configured to provide common mode rejection. The ground electrode prevents power line noise from interfering with the small biopotential signals of interest (e.g., the EEG signals). The location of the ground electrode is not highly correlated to EEG signal quality.

The 10-20 system allows for multiple EEG channels to be recorded across the cortical surface of the subject's brain. This provides the capability to perform mapping and localization of EEG activity using a variety of algorithms. However, many applications utilize only a single EEG channel or a few EEG channels (e.g., 2 or 3). For example, a subject's intrinsic Alpha frequency (IAF) can be determined using EEG data from a single EEG channel. The IAF is the dominant EEG frequency in the EEG Alpha Band. In one example, a repetitive Transcranial Magnetic Stimulation (rTMS) system utilizes the subject's IAF to tailor a magnetic pulse frequency to the subject's brain state. Such personalized rTMS treatment provides additional benefits above standard rTMS on its own.

In some examples, the electrodes in a multi-channel system are contained in a cap that is worn on the head and held in place using straps. An ear clip may be used for the ground or reference electrodes. The cap allows the person to move or shift in their seat without significantly moving the scalp electrodes. However, if one or only a few EEG channels are needed for an application (e.g., determining an IAF), the EEG cap device is unnecessary. In many cases, 10-20 EEG systems are expensive and generally operated by practitioners (e.g., doctors) rather than users themselves. In addition, for such applications, it is unnecessary for the EEG recording electrodes to be positioned in precisely the same place each time the EEG is recorded. For example, a subject's posterior IAF is approximately the same when recorded at P3, P4, Pz, O1, or O2, according to the 10-20 EEG system.

Accordingly, EEG recording systems that are handheld and/or portable are provided herein. In at least one embodiment, the EEG recording system (or device) is positioned approximately at a location (or locations) on the head where the desired EEG characteristic is most evident.

In some examples, the EEG recording system comprises one or more sensing electrodes, one or more reference electrodes, and one or more ground electrodes. The system is configured to be handheld and positioned so that the sensing electrode(s) and reference electrode(s) are on or above the scalp. The sensing electrode(s) and reference electrode(s) are positioned near an area of interest in the brain such that the EEG signal(s) recorded at or near that position will provide necessary information for an application (e.g., determining an IAF). The ground electrode is positioned so that it touches the body of the subject in an area of lower EEG activity.

In some examples, the EEG recording system includes a container (or case), one or more sensing electrodes, and one or more reference electrodes connected to the container. One or more ground electrodes are on the outer surface of the container. The EEG system is configured to be held in place by the subject using their hand(s) such that the sensing electrode(s) and reference electrode(s) are touching the scalp at or near a desired location. Since at least one hand of the subject is touching the ground electrode(s) on the container, a ground path is provided with very little or no EEG activity.

In some examples, the sensing and reference electrodes are dry and/or wet electrodes. Dry electrodes may touch the scalp without the use of a conductive gel. Wet electrodes use a conductive gel between the electrodes and scalp to reduce electrical contact impedance. Dry electrodes comprise short pins, which allow them to penetrate the hair volume and provide solid contact between the tip of the pin and the scalp. Alternately, if the hair volume is not anticipated to interfere with EEG recording, dry electrodes having a metal plate may be used. For example, a metal plate electrode may be used when contacting the subject's forehead or another area where hair growth is not significant. It should be appreciated that other electrode geometries and materials may be used.

In some examples, a ground plate is used as both the reference electrode and the ground connection. In such examples, the electrodes within the container are all sensing electrodes, providing multiple channels of recording.

In some examples, the EEG system includes a ground electrode. The ground electrode provides electrical contact with an area of the person's body which does not generate a significant EEG signal. The ground electrode is used for common mode rejection, generally pertaining to interference from system power lines.

In some examples, the ground electrode includes a flat conductive plate on one side of the container. As such, when the subject holds the EEG system against their scalp, the electrical contact between the subject's fingers and the plate electrode provides a ground connection for the EEG amplifier.

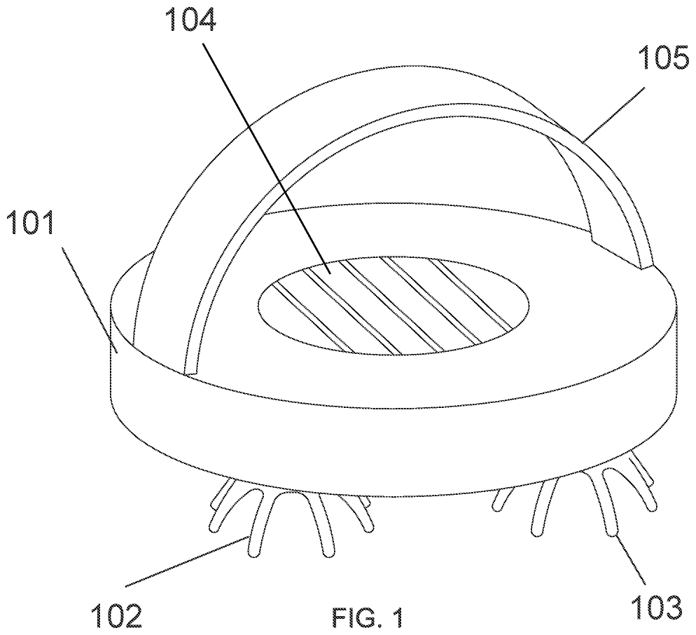

FIG. 1 illustrates an EEG system in accordance with aspects described herein. As shown, the EEG system includes a case (or container) 101, a sensing electrode 102, and a reference electrode 103 on one side of the case 101, and a ground plate 104 on the other side of the case 101. A strap 105 is affixed near opposite edges of the case and allows enough space underneath for a subject (e.g., person) to comfortably insert their fingers (or hand). When the subject's fingers are inserted, they contact the ground plate 104. While the ground plate 104 is shown centered on a surface of the case 101, the ground plate 104 can be positioned at any location where electrical contact is made with the subject. For example, the ground plate 104 (or another ground plate) may be located on a side of the case 101 so that the subject's thumb (or another finger) touches the ground plate 104. In one example, a side ground plate is used in addition to the surface ground plate 104, allowing greater potential to achieve a low contact impedance with one or more ground plates. In some examples, to further reduce the contact impedance between the subject's hand and the ground plate, the subject pre-moistens the part of their hand that makes contact with the ground plate. In some examples, either water or a conductive gel are used to moisten the subject's hand(s). In some examples, the ground plate and/or all electrodes are pre-moistened or coated with conductive gel to further reduce impedance.

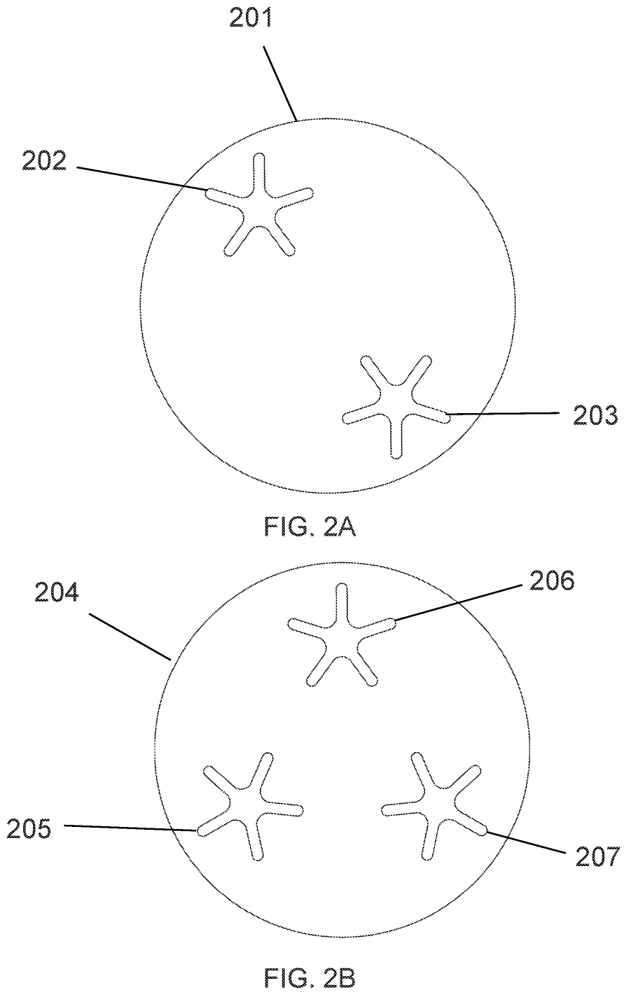

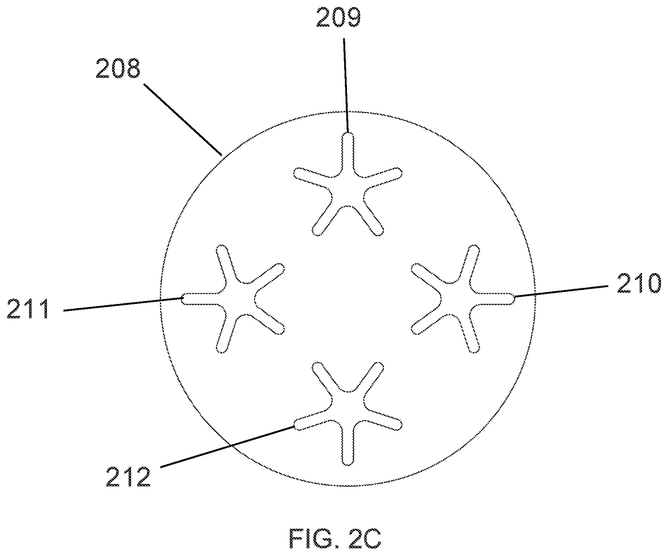

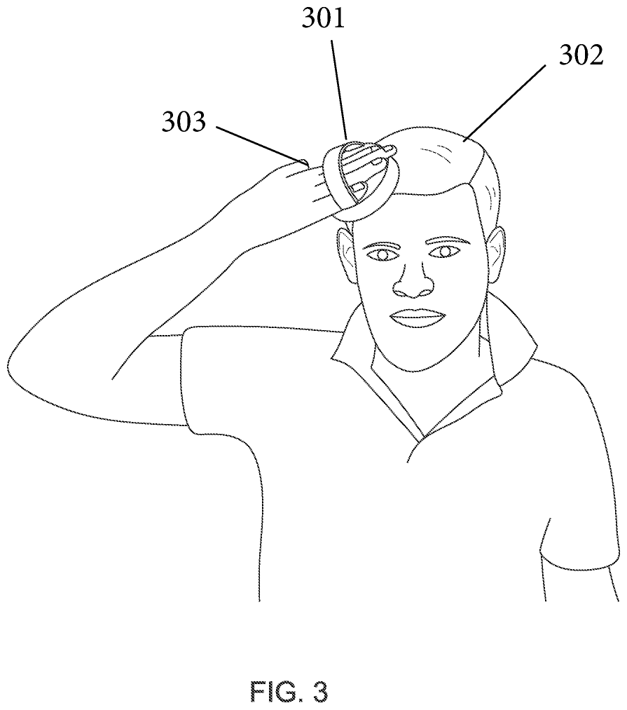

FIGS. 2A-2C illustrate three example configurations of sensing and reference electrodes in accordance with aspects described herein. FIG. 2A shows a bottom surface of a case 201 including a sensing electrode 202 and a reference electrode 203. The sensing electrode 202 and a reference electrode 203 are positioned to record the EEG signal on the surface of the scalp between the two electrodes 202, 203. Alternately, both electrodes 202, 203 could be sensing electrodes, and the reference electrode could be a ground plate electrode (not shown) on the opposite side of the case 201. In this example, the electrode 202 provides one channel of EEG recording and the electrode 203 provides the another channel of EEG recording.

FIG. 2B shows a bottom surface of a case 204 including three electrodes 205, 206, 207. In one example, one of the three electrodes serves as a reference electrode, and the other two electrodes serve as sensing electrodes, providing two separate EEG channels. Alternately, a ground plate (not shown) may serve as the reference, with all three electrodes providing three independent channels of EEG recording.

FIG. 2C shows a bottom surface of a case 208 including four electrodes 209, 210, 211, 212. In one example, one of the four electrodes serves as a reference electrode, and the other three serve as sensing electrodes, providing three separate EEG channels. Alternately, a ground plate (not shown) may serve as the reference, with all four electrodes providing four independent channels of EEG recording.

FIG. 3 shows an example use of an EEG system in accordance with aspects described herein. As shown, a device 301 is held against a subject's scalp 302 in the vicinity of a prespecified location. The person's fingers 303 are positioned to hold the device in place while providing an electrical contact between the person's fingers 303 and the ground electrode plate (not shown) on the device 301. In one example, to record an EEG, the subject is instructed to relax, close their eyes, and hold the device as still as possible during recording. In some examples, the EEG recording is used to the calculate the subject's IAF.

If the EEG recording is to be performed for an extended period of time, or if the subject is unable to hold the device (e.g., device 301) in place, then a headband may be incorporated into the device. The center of the forehead (FPz) is generally considered to be an area of lower EEG activity and is often used as a ground electrode location. In some examples, the headband comprises a plate electrode that is electrically connected to the plate electrode on the device. The headband may be permanently incorporated into the device or it may be removable.

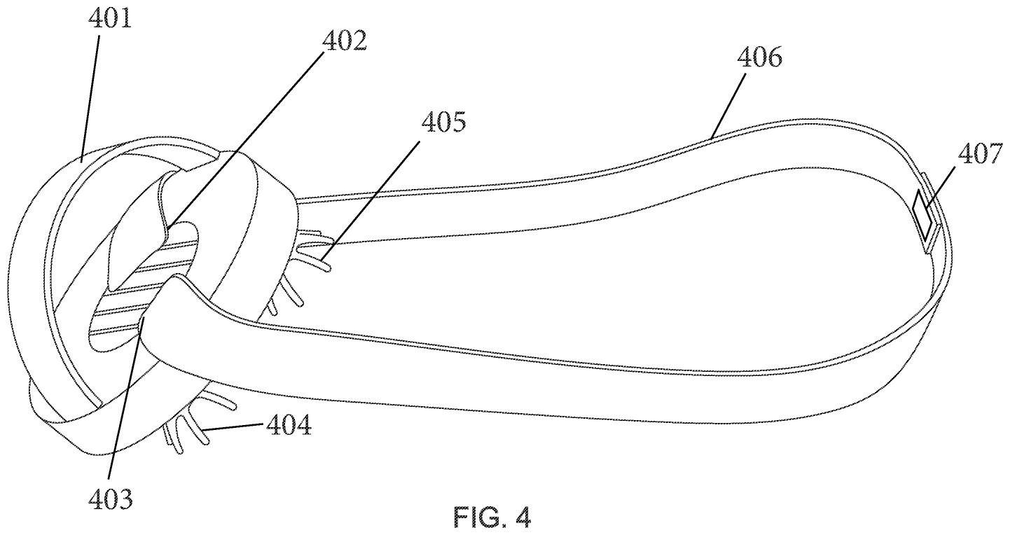

FIG. 4 illustrates an example of a headband EEG system in accordance with aspects described herein. As shown, the device 401 comprises a headband 406 that is connected to the plate electrode at two locations 402, 403. In some examples, a wire is incorporated into the headband (not shown). The wire (or wires) electrically connects the plate electrode 407 to the ground plate. In some examples, the plate electrode 407 serves as a ground for the system (e.g., device 401). On the opposite side of the device 401, two electrodes 404, 405 are used to record the EEG of the subject. In some examples, the electrode 404 is a sense electrode and the electrode 405 is a reference electrode, or vice versa.

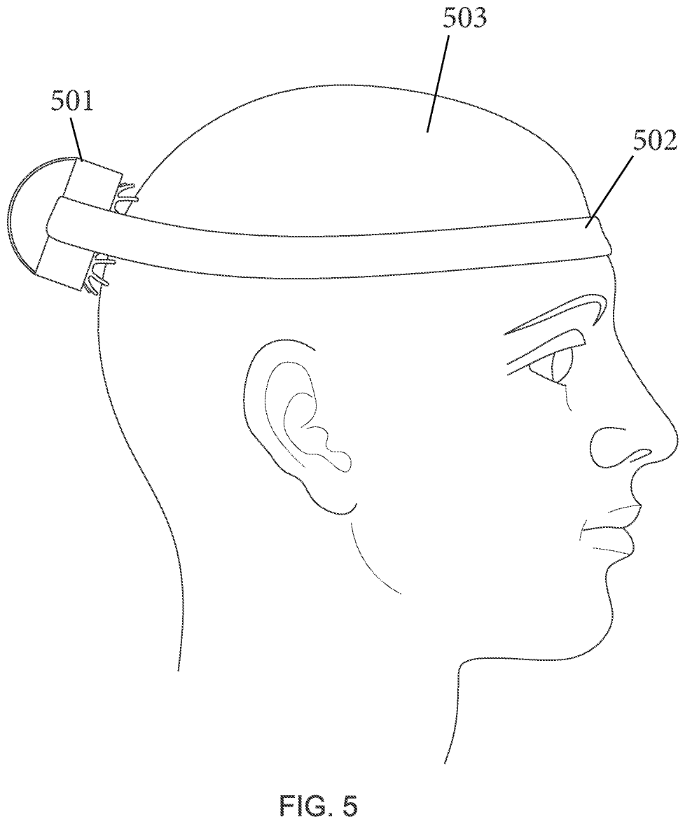

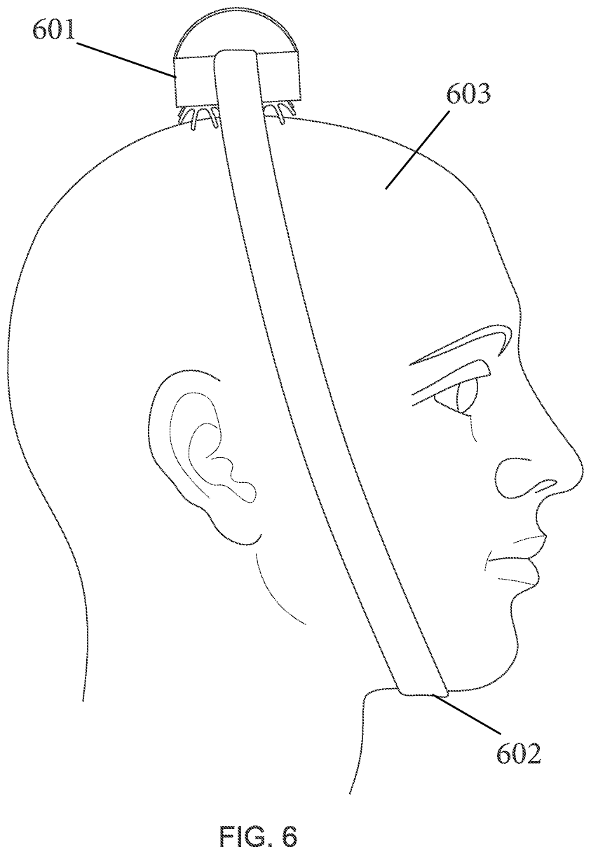

FIG. 5 illustrates an example of a headband EEG system 501 in accordance with aspects described herein. As shown, the device 501 is configured to be worn on a subject's head 503. In some examples, the sense and reference electrodes of the device 501 touch the posterior region of the subject's head 503. A ground plate electrode (not shown) is positioned on the underside of the headband 502. The ground plate presses against the subject's forehead, serving as a ground electrode for the EEG system.

FIG. 6 illustrates another example of a headband EEG system in accordance with aspects described herein. As shown, the device 601 is held against the top of the subject's head 603 with a headband 602. In some examples, the headband 602 is held under the chin of the subject. The ground plate electrode (not shown) touches the underside of the chin of the subject, which is another area with very little EEG electrical activity. In some examples, the subject is instructed to refrain from speaking or swallowing while recording an EEG to minimize muscle artifact in the EEG signal.

As described above, the headband EEG systems of FIGS. 4-6 allow the subject to hold the device in place, even if the person is unable to use their fingers (or hand) to press the device in place (e.g., as shown in FIG. 3). Alternatively, two straps may comprise handgrips that allow the subject to hold the device in place while pulling down on the straps. In some examples, one or both straps include a plate electrode to provide an electrical connection between the subject's hand(s) and the ground plate on the device.

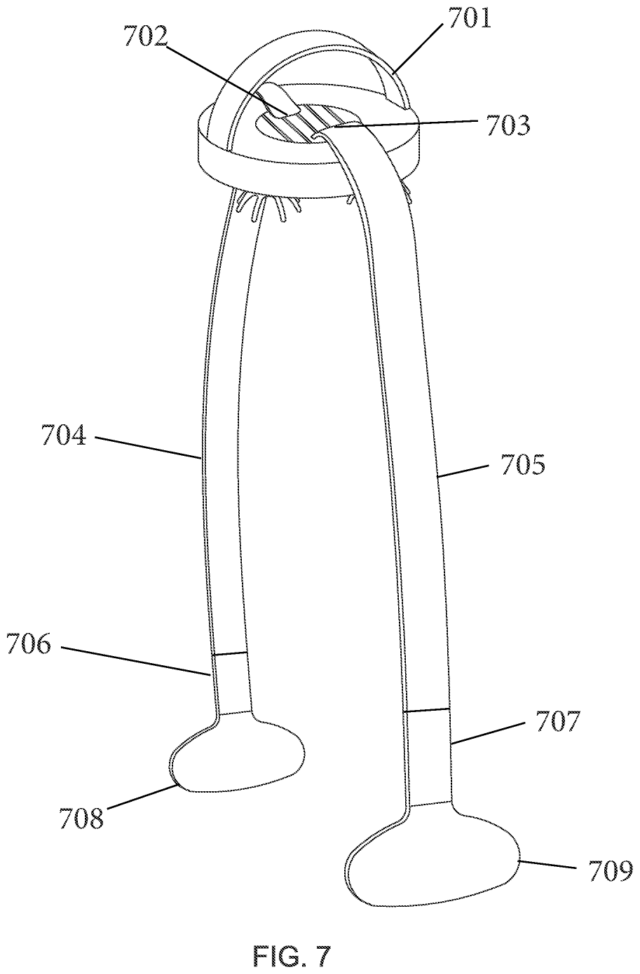

FIG. 7 shows an example of a strap EEG system in accordance with aspects described herein. As shown, the device 701 comprises two straps 704, 705 that connect to the device plate electrode at two locations 702, 703. Two plate electrodes 706, 707 are positioned near the end of the straps. Handgrips 708, 709 are located at the end of the straps such that the subject's hands do not slip off the straps when the device 701 is being held in place (e.g., on the subject's scalp).

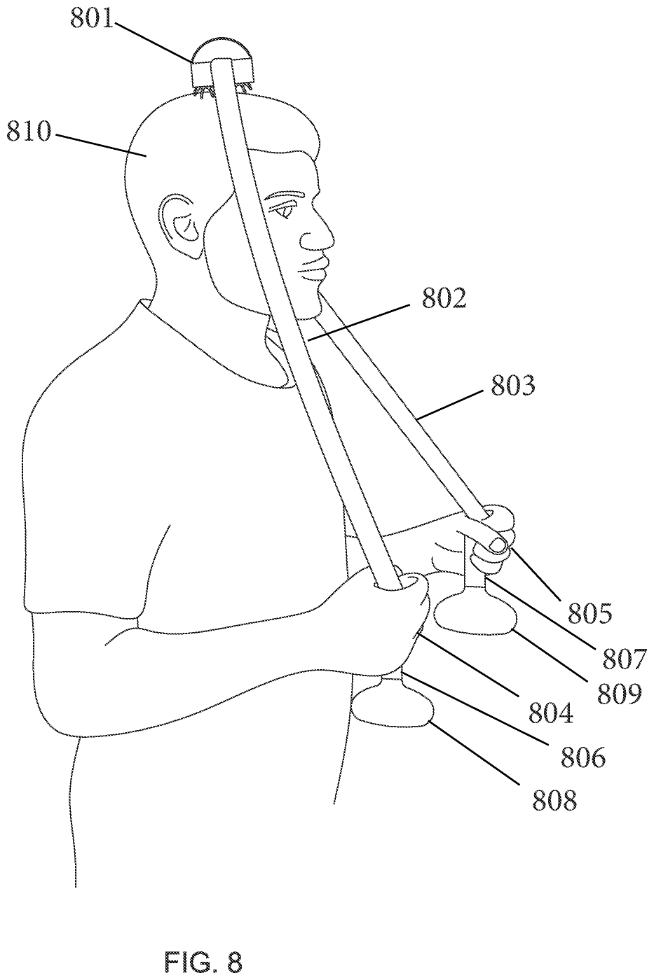

FIG. 8 illustrates an example of a subject holding a strap EEG system in accordance with aspects described herein. As shown, the device 801 is held against the subject's scalp 810 using two straps 802, 803. The subject grips the plate electrodes 806, 807 with their hands 804, 805 to make an electrical connection. The wide areas at the ends of the straps 808, 809 ensure that the subject's hands 804, 805 do not slip off the straps during recording.

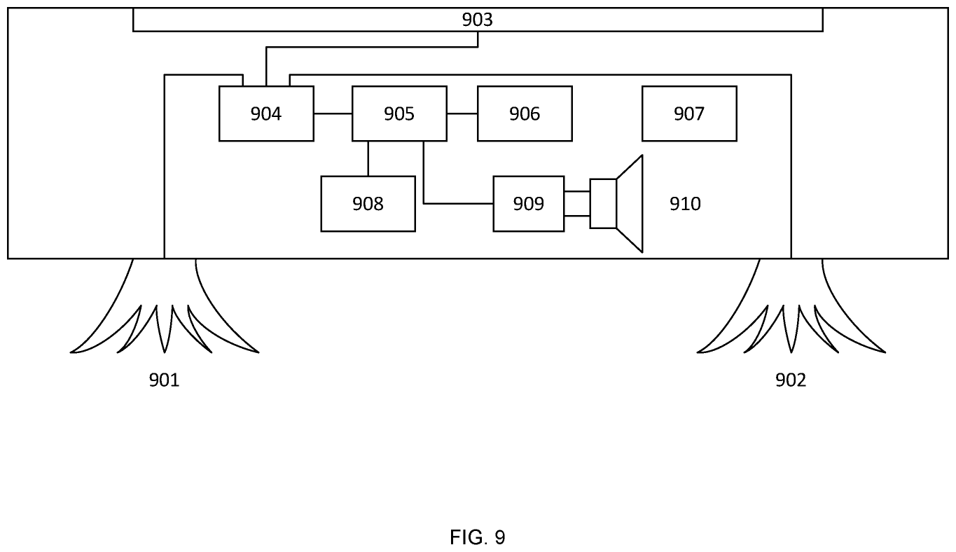

FIG. 9 illustrates an example internal configuration of an EEG system in accordance with aspects described herein. A sensing electrode 901 and a reference electrode 902 are attached to one side of the device. A ground electrode plate 903 is attached to the opposite side of the device. The three electrodes are connected to an EEG amplifier 904, which amplifies the EEG signal and sends the output to a processor 905. In some examples, the processor includes an Analog-to-Digital Converter (ADC). In some examples, the ADC is a separate component coupled to the processor 905. The processor 905 uses memory 906 for program and data storage.

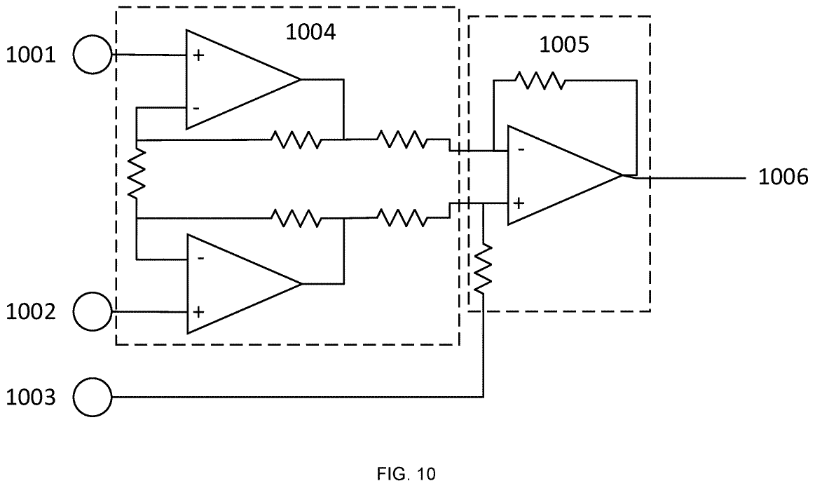

FIG. 10 illustrates an example EEG amplifier in accordance with aspects described herein. In some examples, the EEG amplifier corresponds to the EEG amplifier 904 of FIG. 9. The EEG amplifier uses the input between a sense electrode 1001 and a reference electrode 1002, with a ground electrode 1003 serving to remove any common mode noise. A differential amplifier 1004 is used to amplify and isolate the signal between the two electrodes 1001, 1002. The output of the differential amplifier 1004 is sent to a buffer 1005, which removes the common mode noise. In some examples, the buffer output 1005 is sent as an output 1006 to an ADC (e.g., an ADC incorporated into the processor 905 of FIG. 9).

In some examples, the device is powered using a battery 907. The battery may be a primary cell, such as a disposable battery, or the battery may be rechargeable. Alternately, the device is configured to use an external power source. In some examples, the processor 905 performs calculations automatically and produces a result (e.g., an IAF). In some examples, the processor 905 allows the transmission of EEG data (or a corresponding result or calculation), either from memory or streaming. In such examples, a wireless communications module 908 is used to send data to an external device, such as a computer or mobile device, such as a cell phone. Wireless communication is achieved through Bluetooth, Wi-Fi, RF, or any other suitable means. Alternately, a wired communication system is used, where the device is connected to an external device using a cable (e.g., USB, Ethernet, etc.).

In some examples, an audio amplifier 909 connects to the processor 905 and sends an audio signal to a speaker 910. The audio signal provides status information to the user, such as the subject, a caregiver, doctor, etc. The status information provides an indication of electrical impedance between the two electrodes, EEG quality, ground connectivity, battery life, communications link, or some other information. In some examples, the audio signal provides an indication of the user's alignment with a desired brain region. The EEG device can include one or more positional sensors (e.g., an accelerometer, a gyroscope, etc.) that provide an indication of the EEG device relative to the different brain regions. In some examples, the audio signal provides feedback to the user based on the collected positional data to guide the user to the desired brain region.

In some examples, the processor 905 communicates with an external device (e.g., a computer or mobile phone) to assist the user with alignment. In some examples, a camera of an external device is used to provide feedback to the processor 905. Using the external camera, image processing and object detection techniques are used to map the location of the EEG device relative to different brain regions. In some examples, the external camera data is used in combination with the positional sensor data to map the location of the EEG device. Visual cues are provided via the external device to guide the user to the desired brain region. Alternatively, the audio signal provided by speaker 910 of the EEG device provides the position feedback to the user.

In some examples, the electrodes 901, 902 are configurable by the user (e.g., the subject, a caregiver, etc.). The user can select a function (e.g., sensing or reference) for each electrode. In some examples, the user configures the electrodes using an application on an external device connected to the EEG system, such as a computer or mobile device. In some examples, the EEG system includes a user interface (e.g., buttons, switches, touchscreen, etc.) that allows the user to configure the electrodes. The processor 905 is configured to implement the assigned functions of each electrode (e.g., sensing or reference). In some examples, the processor 905 operates one or more electrical switches within the EEG system to assign each role to the electrodes 901, 902. In some examples, the switches are operated to connect the sensing electrode(s) or the reference electrode(s) to specific input or output paths of the processor 905. In some examples, the electrodes are automatically configured (and reconfigured) by the EEG system or an external device connected to the EEG system. By reconfiguring the electrodes, the EEG device can collect EEG recordings from different areas of the brain without significant movements or adjustments from the user (e.g., the subject). In some examples, the EEG system reconfigures the electrodes while being held in the same location by the user. In such examples, the EEG system can collect slightly different EEG recordings around a common brain region. The different EEG recordings are used to validate the resulting subject metrics (e.g., IAF).

As described above, the EEG recording system (or device) is used to record EEG data at specific locations on the head where desired EEG characteristic are most evident. In some examples, the desired EEG metrics or characteristics are calculated from a single EEG recording (e.g., a 30 second recording, a 1 min recording, etc.). In some examples, the subject is instructed to collect multiple EEG recordings from the same location. The desired EEG metrics or characteristics are calculated for each EEG recording and averaged together to derive the final EEG metrics or characteristics. In some examples, the collection of multiple EEG recordings minimizes any inconsistencies in the recordings due to unsupervised collection (e.g., without the supervision of a practitioner).

In some examples, the subject is instructed to collect multiple EEG recordings from different locations. For example, the subject may collect one or more EEG recordings from the P3 region during a first period of time, one or more EEG recordings from the Pz region during a second period of time, and one or more EEG regions from the O1 region during a third period of time. The EEG system (or an external device) combines the recordings from the different regions to produce an aggregated EEG recording. In some examples, the aggregated EEG recording mimics an EEG recording from a 10-20 EEG system (or a cap system). In some examples, a time shift and/or phase shift is applied to one or more of the recordings to produce the aggregated EEG recording. In some examples, one or more transform functions are used to produce the aggregated EEG recording.

While the examples above describe collecting EEG data to determine the subject's IAF, it should be appreciated that different EEG metrics and characteristics can be determined. For example, the EEG data can be used to determine an Alpha Frequency Variability (AFV) of the subject. In addition, the EEG data can be used to determine EEG metrics and characteristics for any band, including the Delta band (0.5 Hz-3 Hz), the Theta band (3 Hz-8 Hz); the Alpha band (8 Hz-13 Hz), the Beta band (13 Hz-38 Hz), and the Gamma band (38 Hz-42 Hz).

In some examples, the EEG metrics and characteristics are used to provide treatment (e.g., rTMS) to the subject via at least one treatment device. The treatment device is configured to provide treatment (e.g., brain stimulation) to the subject's brain based on the EEG metrics and characteristics of the subject. In some examples, the treatment device is configured to provide magnetic brain stimulation to optimize at least one EEG metric or characteristic. In some examples, the treatment corresponds to a treatment plan (or treatment settings). In some examples, the treatment device includes at least one magnetic source (e.g., electromagnet or permanent magnets) configured to provide at least one magnetic field having a stimulation (or therapeutic) frequency. In some examples, the treatment device includes at least one rotating magnet. In some examples, the treatment device corresponds to one or more devices, mechanisms, and techniques described in U.S. Pat. No. 9,308,387, titled “Systems and Methods for Neuro-EEG Synchronization Therapy” and granted on Apr. 13, 2016, which is hereby incorporated by reference herein in its entirety.

In some examples, the treatment device includes at least one stimulation source (e.g., magnetic source, electric source, etc.). The at least one stimulation source provides, at least in part, a stimulation treatment at one or more stimulation frequencies. In some examples, the treatment device provides brain stimulation using transcranial electrical stimulation. In some examples, the treatment device provides brain stimulation using focused ultrasound. In some embodiments, the treatment device provides brain stimulation using functional Near Infrared Spectroscopy (fNIRS). In some examples, the treatment device provides brain stimulation using sensory stimulation. For example, in some embodiments the treatment device provides sensory stimulation including flashing light, sound, video, or touch.

What is claimed is:

1. A device for measuring electroencephalography (EEG) recordings of a subject, comprising:
a casing;
at least two first electrodes extending from a first surface of the casing, the at least two first electrodes being configured to contact a scalp of the subject during an EEG recording; and
at least one second electrode disposed on a second surface of the casing and configured to provide a ground connection during the EEG recording.

2. The device of claim 1, wherein the at least two first electrodes includes at least one sensing electrode and at least one reference electrode.

3. The device of claim 2, wherein the at least one sensing electrode is configured to contact the scalp at a desired location for the EEG recording.

4. The device of claim 2, wherein the at least one reference electrode is configured to contact the scalp at a location of low electrical activity.

5. The device of claim 1, wherein the second surface of the casing is opposite the first surface of the casing.

6. The device of claim 1, wherein the at least one second electrode is configured to contact a hand of the subject while the subject is holding the device.

7. The device of claim 6, further comprising a strap connected across the second surface of the casing, wherein the strap allows the hand of the subject to hold the device while contacting the at least one second electrode.

8. The device of claim 1, further comprising a strap including a third electrode and at least one wire electrically connected to the third electrode.

9. The device of claim 8, wherein the at least one wire is electrically connected to the at least one second electrode forming an electrical connection between the at least one second electrode and the third electrode.

10. The device of claim 9, wherein the strap is configured to be worn such that the third electrode contacts the subject's forehead.

11. The device of claim 9, wherein the strap is configured to be worn such that the third electrode contacts the subject's chin.

12. The device of claim 1, further comprising:
a first strap connected to the second surface of the casing; and
a second strap connected to the second surface of the casing,
wherein the first and second straps are configured to be pulled on by the subject to hold the device against the scalp of the subject.

13. The device of claim 12, wherein one of the first strap and the second strap includes a third electrode and at least one wire electrically connected to the third electrode, wherein the at least one wire is electrically connected to the at least one second electrode forming an electrical connection between the at least one second electrode and the third electrode.

14. The device of claim 13, wherein a hand of the user contacts the third electrode while pulling on the first strap or the second strap.

15. The device of claim 1, further comprising:
a first strap including a third electrode and at least one first wire electrically connected to the third electrode; and
a second strap including a fourth electrode and at least one second wire electrically connected to the fourth electrode.

16. The device of claim 15, wherein the at least one first wire is electrically connected to the at least one second electrode forming an electrical connection between the at least one second electrode and the third electrode, and the at least one second wire is electrically connected to the at least one second electrode forming an electrical connection between the at least one second electrode and the fourth electrode.

17. The device of claim 16, wherein the first and second straps are configured to be pulled on by the subject to hold the device against the scalp of the subject.

18. The device of claim 16, wherein a first hand of the user contacts the third electrode while pulling on the first strap and a second hand of the user contacts the fourth electrode while pulling on the second strap.

19. The device of claim 1, further comprising at least one audio speaker configured to provide audio feedback to the subject.

20. The device of claim 19, wherein the audio feedback provides an indication that the EEG recording has started and/or an indication that the EEG recording has ended.

21. The device of claim 19, wherein the audio feedback provides an indication of the device's location relative to a desired location on the scalp of the subject.

22. The device of claim 1, further comprising at least one battery.

23. The device of claim 22, wherein the at least one battery is rechargeable.

24. The device of claim 1, further comprising at least one processor.

25. The device of claim 24, wherein the at least one processor is configured to send data associated with the EEG recording to at least one external device.

26. The device of claim 25, wherein the at least one processor is configured to determine at least one EEG characteristic of the subject based on the EEG recording.
